(19) United States
(12) Patent Application Publication
(10) Pub. No.: US 2007/0250011 A1
(43) Pub. Date: Oct. 25, 2007

(54) Winged venous catheter

(76) Inventor: David Lee, Taipei City (TW)

Correspondence Address: BIRCH STEWART KOLASCH & BIRCH, PO BOX 747, FALLS CHURCH, VA, 22040-0747, US

(21) Appl. No.: 11/394,093

(22) Filed: Mar. 31, 2006

(51) Int. Cl. A61M 5/178

(52) U.S. Cl. 604/165.03

(57) ABSTRACT

A winged venous catheter capable of simplifying a medical treatment procedure for medical professionals includes a barrel having a set of fixed wings, a soft tube having a tapered diameter and extended from an end of the barrel, a connecting opening extended from another end of the barrel without the soft tube. The barrel includes a position limiting portion disposed on an internal wall and coupled to the soft tube for preventing accidental pierces. The invention provides medical professionals to insert a syringe needle into the soft tube through the barrel, use the clamped fixed wing to insert the syringe needle into the vein, and pull the syringe needle out from the vein but let the soft tube remain in the vein for facilitating liquid medicines to continuously flow from the connecting opening into the vein through the soft tube.

### FIELD OF THE INVENTION

The present invention relates to a venous catheter, and more particularly to a venous catheter having a set of fixed wings.

### BACKGROUND OF THE INVENTION

The purpose of an intravenous injection is to replenish body fluids, electrolytes and nutrients or to provide medicines to patents for medical treatments, and the intravenous injection is a very important treatment for severely ill patients. A syringe needle for intravenous injections is disclosed in R.O.C. Pat. Publication No. 570825 entitled “Needle seat of a safety syringe”, and the syringe needle is connected to a needle seat. Obviously, such hard syringe needle is very different from the soft bendable property of the veins, and thus continuous treatments for patients by using the syringe needle for intravenous injections cannot be achieved.

R.O.C. Pat. No. M274966, entitled “Safety device of a winged syringe”, comprises a winged syringe, a needle cap, and a safety clamp, wherein a cannula is connected at a rear end of the winged syringe, and the syringe needle of the winged syringe is shorter than the syringe needle of the foregoing safety syringe needle. A patient will feel a little pain when the winged syringe is inserted into the patient's vein, but the winged syringe can be retained for several days of continuous treatments and injected directly with the required medicine from the tube connected to the winged syringe. Further, medical professionals can hold both fixed wings of the winged syringe to insert the syringe needle quickly into the vein. Although the syringe needle of the winged syringe is shorter, the hard syringe needle retained in the vein still has the risks of breaking the veins or leaking the liquid infusion due to the shaking and movements of the patient. On the other hand, the fixed wings fixed at the position of the vein by an adhesive tape may affect the blood circulation due to the long-time pressing, and thus having the danger of causing phlebitis.

To avoid the breaking of veins or the leaking of liquid infusions, a venous catheter is installed into the winged syringe, and the venous catheter can reduce the number of times for piercing blood vessels, the pain of patients, the damage to blood vessels and the leakage of fluid infusion and the venous catheter also can effectively protect blood vessels. Therefore, it is advantageous to use venous catheters for clinical medications and emergency rescues and lower the possibility of phlebitis. R.O.C. Pat. Publication No. 425922, entitled “venous catheter structure”, disclosed an integrated tubular structure having a hard tube disposed at its top, a wing protruded from an external wall of the tube, a soft bendable cannula disposed at the bottom of the tube, and the bottom of the cannula is substantially in a conical shape. Although the soft cannula is flexible and can be bent according to the vein to avoid piercing the vein, the volume of the venous catheter of this kind is smaller, and thus medical professionals cannot sheath the syringe needle for injecting medicine into the vein directly and need an additional syringe to provide sufficient space for medical professionals to inject medicines. However, the many-layer assembling procedure usually delays the prime time of the rescue. Further, the syringe occupies some space, such that the syringe becomes an obstacle of medical treatment when an emergency occurs or the patient is unconscious.

### SUMMARY OF THE INVENTION

The primary objective of the present invention is to overcome the foregoing shortcomings and avoid the existing deficiencies by providing a winged venous catheter that can simplify the medical treatment procedure and reduce the damages to the veins, and the winged venous catheter comprises: a barrel having a set of fixed wings, a folding portion and a frictional position disposed on the fixed wings to facilitate medical professionals to use the syringe for injections, a soft pad for protecting blood vessels from being pressed by the syringe for a long time, a soft tube having a tapered diameter and a soft bendable properties and extended from an end of the barrel, and a safety casing extended from the barrel and sheathed onto an end of the soft tube, wherein the barrel includes a connecting opening extended from another end of the barrel for inserting the syringe needle, and a position limiting portion disposed at an internal wall of the barrel and coupled to the soft tube for preventing a pierce by mistake, and the position limiting portion can be used to guide the syringe needle into the soft tube.

Medical professionals can hold the fixed wings to insert the syringe needle connected to the soft tube into patient's vein, and then draw the syringe needle out from the vein but leave the soft tube of the winged venous catheter in the vein for facilitating liquid medicines to flow from the connecting opening into the vein from the soft tube. From these steps, we know that the winged venous catheter of the invention allows medical professionals to apply medicines quickly and accurately into the vein of a severely or critically ill patient for a liquid infusion treatment, and improve the efficiency and quality of medical treatments and shorten the rescue time.

### BRIEF DESCRIPTION OF THE DRAWINGS

FIG. 1 is a perspective view of the present invention;

FIG. 2 is a cross-sectional view of the present invention;

FIG. 3 is a perspective view of a first preferred embodiment of the present invention;

FIG. 4 is a perspective view of a second preferred embodiment of the present invention;

FIG. 5 is a perspective view of a third preferred embodiment of the present invention; and

FIG. 6 is a schematic view of using the present invention.

### DETAILED DESCRIPTION OF THE PREFERRED EMBODIMENTS

The present invention will now be described in more detail hereinafter with reference to the accompanying drawings as follows.

The invention discloses a winged venous catheter that can be bent according to a patient's vein and avoid piercing the vein, so that medical professionals can quickly and accurately inject fluid medicines into the patient's vein.

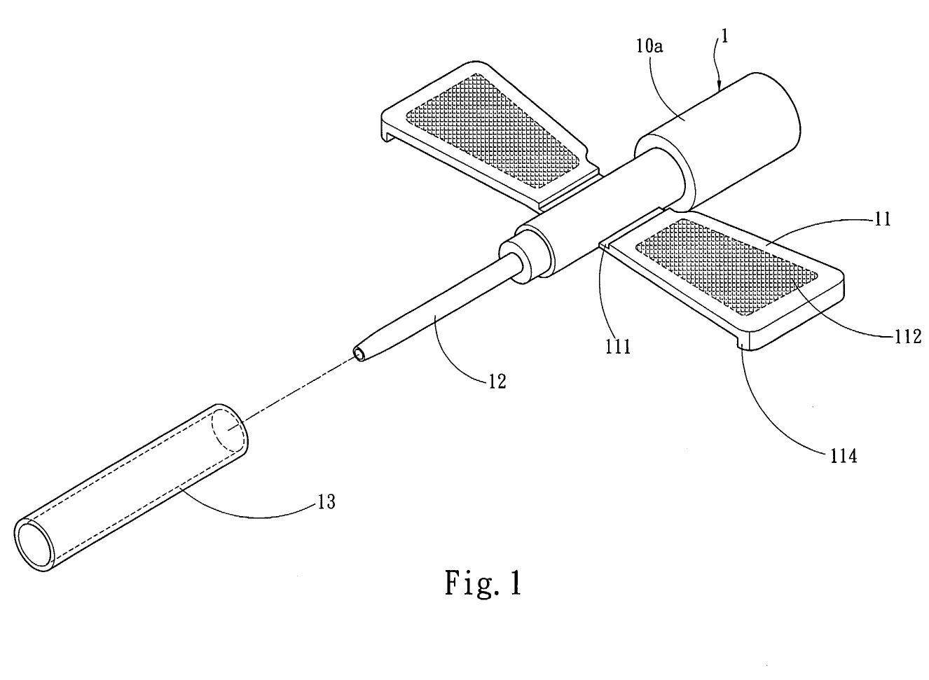

Referring to FIG. 1 for the perspective view of the present invention, the winged venous catheter 1 of the invention comprises a barrel 10a having a set of fixed wings 11, a folding portion 111 and a frictional portion 112 disposed on the fixed wings 11 for facilitating medical professionals to hold the fixed wings 11 for an injection. Further, each fixed wing 11 includes a soft pad 114 for protecting the blood vessel from being pressed for a long time. A soft tube 12 having a tapered diameter and a soft bendable property is extended from an end of the barrel 10a. Further, the barrel 10a has a safety casing 13 extended from an end of the barrel 10a and sheathed onto the soft tube 12 for protecting the soft tube 12.

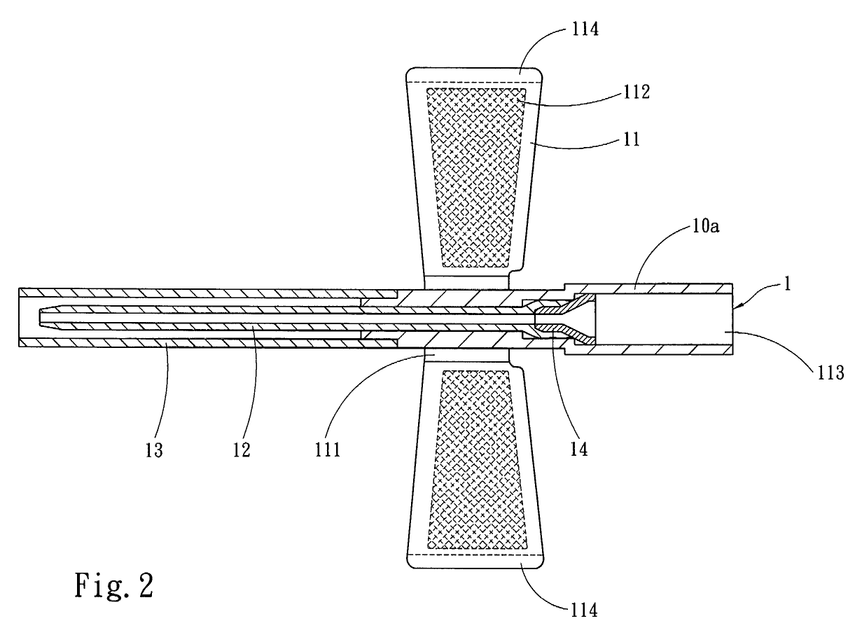

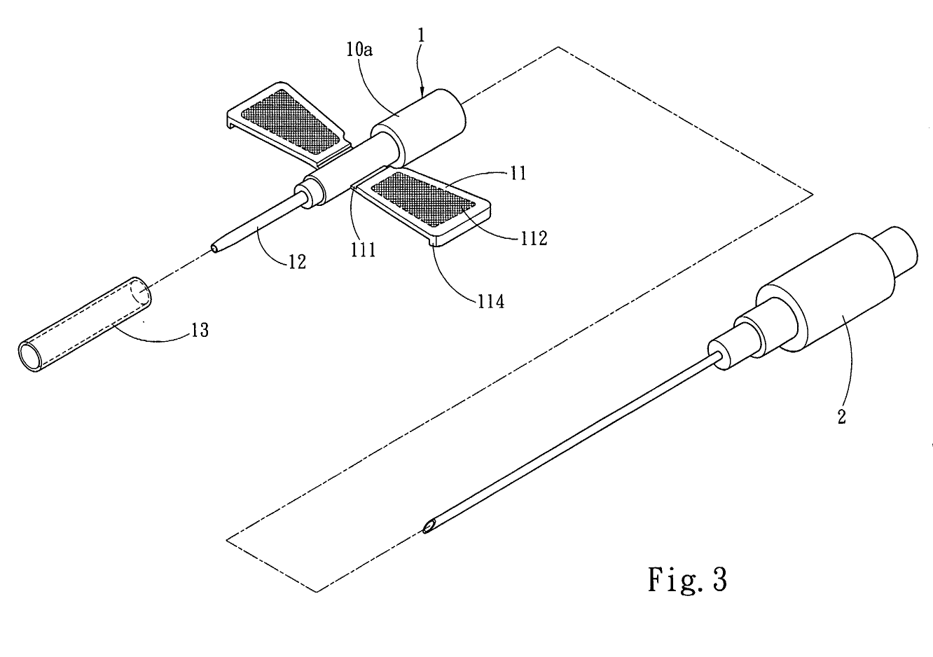

Referring to FIG. 2 for the cross-sectional view of the present invention, the barrel 10a has a connecting opening 113 extended from another end without the soft tube 12 of the barrel 10 and a position limiting portion 14 disposed on an internal wall of the barrel 10a and coupled to the soft tube 12 for preventing the syringe needle 2 (as shown in FIG. 3) from piercing people by mistake and for guiding the syringe needle 2 into the soft tube 12.

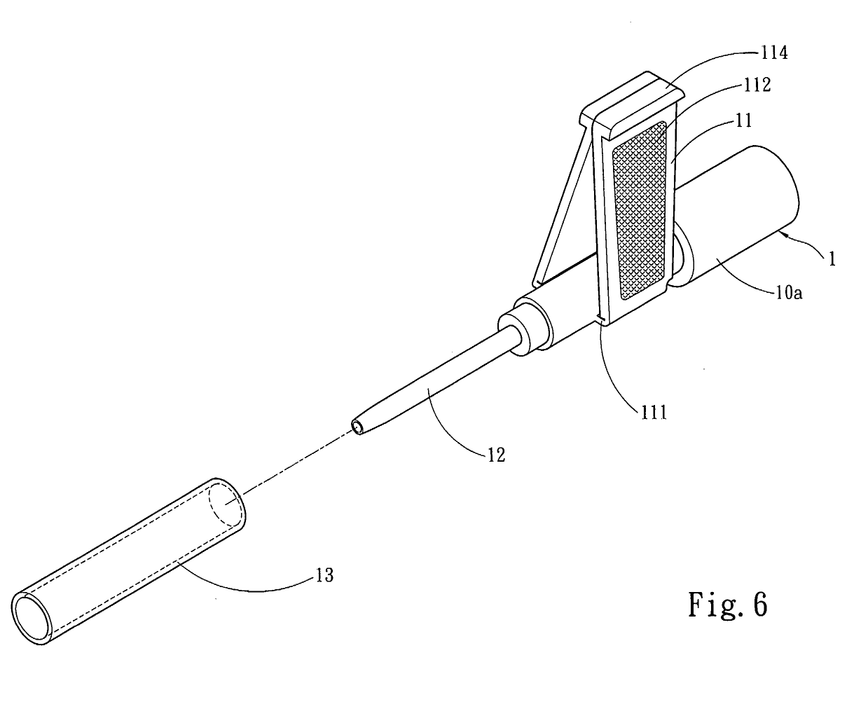

Referring to FIGS. 3 and 6 respectively for the perspective view and schematic view of using the invention according to a preferred embodiment, medical professionals sheathe the barrel 10a and pass it into the connecting opening 113 of syringe needle 2, and thus the folding portion 111 clamps the two fixed wings 11 towards a sunken position. Now, medical professional's hands touch the frictional section 112, and then pull out the safety casing 13 that is used for avoiding accidental pierces while the syringe needle 2 is pulled out from the soft tube 12, and then the syringe needle 2 connected to a soft tube 12 is inserted into the vein, and the syringe needle 2 is pulled out from the vein, but the soft tube 12 of the winged venous catheter 1 remains in the vein to facilitate flowing the liquid medicines from the connecting opening 113 into the vein through the soft tube 12. A tape is used for attaching the fixed wing 11 with a side of the soft pad 114 onto the patient's skin. With the soft pad 114, the fixed wing 11 is heightened, so that the fixed wing 11 maintains a distance apart from the surface of skin to reduce the area of the fixed wing 11 compressing blood vessels that will affect blood circulation.

It is worth to note that the position limiting portion 14 of the barrel 10a can quickly and accurately guide the syringe needle 2 into the soft tube 12, since the slanting side of the position limiting portion 14 can correct the syringe needle 2 to the center of the barrel 10a and pass through the soft tube 12 to enter into the safety casing 13.

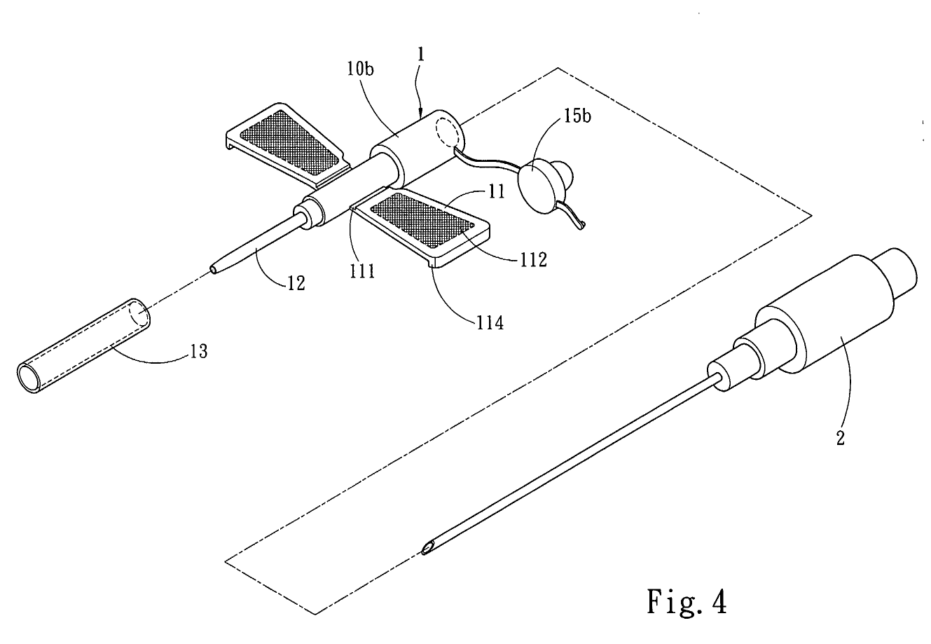

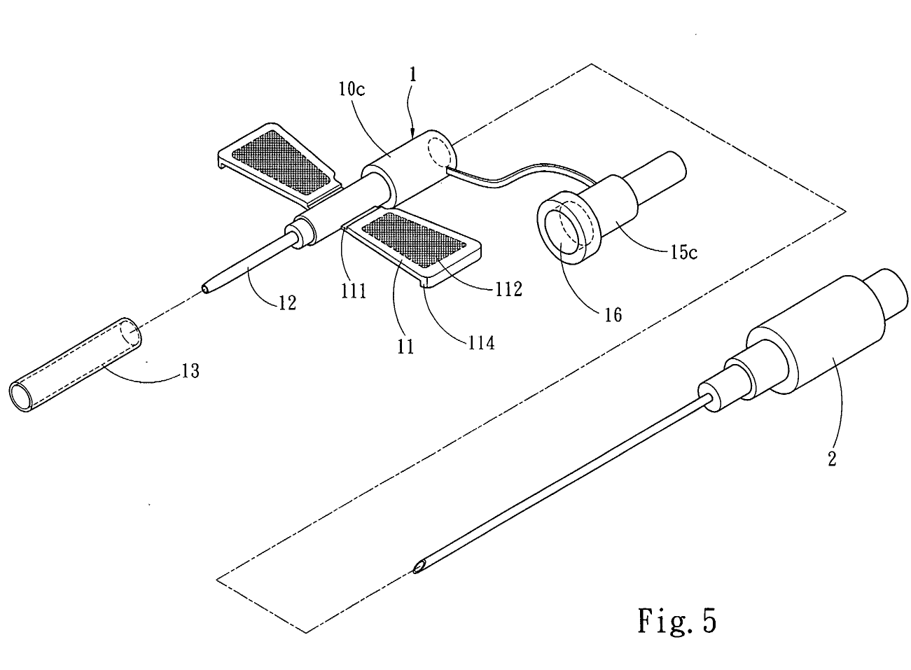

The barrel 10b of the winged venous catheter 1 can come with a plug 15b (as shown in FIG. 4) as a device for sealing the connecting opening 113 when the liquid infusion is suspended, so as to prevent the cut from being infected, or the barrel 10c comes with a cannula 15c having a rubber 16 (as shown in FIG. 5), and the cannula 15c can be inserted into the connecting opening 113. Such arrangement not only seals the connecting opening 113, but also inserts the syringe (not shown in the figure) into the rubber 16 for the necessary procedure, so as to simplify the medical treatment steps.

While the invention has been described by means of specific embodiments, numerous modifications and variations could be made thereto by those skilled in the art without departing from the scope and spirit of the invention set forth in the claims.

What is claimed is:

1. A winged venous catheter, comprising:
a barrel, having a set of fixed wings, a soft tube extended from an end of said barrel, and a connecting opening disposed at another end of said soft tube of said barrel, and the internal wall of said barrel including a position limiting portion connected to said soft tube for preventing a pierce by mistake.

2. The winged venous catheter of claim 1, wherein said fixed wing includes a folding portion.

3. The winged venous catheter of claim 1, wherein said fixed wing includes a frictional portion.

4. The winged venous catheter of claim 1, wherein said fixed wing includes a soft pad.

5. The winged venous catheter of claim 1, wherein said barrel includes a soft tube having a tapered diameter and extended from an end of said barrel.

6. The winged venous catheter of claim 1, wherein said barrel includes a safety casing sheathed onto an end of said soft tube.

7. The winged venous catheter of claim 1, wherein said barrel includes a plug.

8. The winged venous catheter of claim 1, wherein said barrel includes a cannula having a rubber.
